(19) United States
(12) Patent Application Publication
(10) Pub. No.: US 2026/0069328 A1
(43) Pub. Date: Mar. 12, 2026

(54) FLEXIBLE DEVICE FOR TREATING A BONE FRACTURE

(71) Applicant: I.T.S. GmbH, Lassnitzhöhe (AT)

(72) Inventor: Ronald PRAGER, Bovenau (DE)

(73) Assignee: I.T.S. GmbH, Lassnitzhöhe (AT)

(21) Appl. No.: 19/134,008

(22) PCT Filed: Jan. 29, 2024
(86) PCT No.: PCT/AT2024/060022

§ 371 Date: May 29, 2025

(30) Foreign Application Priority Data
Apr. 21, 2023 (AT) A 50299/2023

(51) Int. Cl. A61B 17/74; A61B 17/72

(52) U.S. Cl. CPC A61B 17/744; A61B 17/7241

(57) ABSTRACT

A device for treating a bone fracture, in particular a femur fracture such as a femoral neck break, includes a holding part fastenable in or on a first bone part, wherein the holding part has at least one opening through which a bone screw and an adjusting screw are guided. The opening includes on the inner side a surface having a threaded surface that the adjusting screw engages, with the bone screw being able to slide freely in the opening, and the adjusting screw is configured to allow the bone screw to rest against at least one stop of the adjusting screw, if necessary after a lateral sliding of the bone screw. An adapter that can be fixed in the bone screw to be laterally releasable forms a barrier for the adjusting screw in the event of lateral movement of the adjusting screw.

The invention relates to a device for treating a bone fracture, in particular a femur fracture such as a femoral neck break, comprising a holding part which can be fastened in or on a first bone part, wherein the holding part has at least one opening through which a bone screw and an adjusting screw are guided, wherein the opening comprises on the inner side a surface having a threaded surface in which the adjusting screw engages, with the bone screw being able to slide freely in the opening, and the adjusting screw is configured to allow the bone screw to rest against at least one stop of the adjusting screw, if necessary after a lateral sliding of the bone screw.

Femur fractures, in particular femoral neck breaks, are fractures, the proper treatment of which is often difficult. With femoral neck breaks, it is in particular also the case that fractures of this type occur more prevalently in older, often elderly people, which further complicates a good treatment. Femoral neck breaks are frequently the result of a bone structure which is no longer fully intact, for example due to progressing osteoporosis.

For the treatment of femur fractures, one difficulty is that the broken bone parts must be brought into a suitable position relative to one another and are to be fixed in said position at least immediately following the treatment. This is also rendered more difficult in that, due to the bone break, the muscles surrounding the bone are not in an ideal position and that, therefore, this muscular component must also be taken into consideration in relation to tensile and compressive forces, but also any torsion forces that may occur, which are to be prevented.

In the treatment of femoral neck breaks, holding parts such as nails or plates which have openings are used, through which openings a bone screw is guided which, after a previous drilling, runs through a first, lateral bone part and is screwed into a second, medial bone part that has been separated from said first bone part as the result of a break. In order to move the bone screw after it has been screwed into the second, medial bone part and to thereby achieve a positioning of the second bone part against the first bone part, it has become known from the prior art to use a compression screw which engages in a thread that runs on a side of the bone screw and interacts with said bone screw. By screwing in the compression screw adjacently to the bone screw, it is possible to initially screw in the compression screw until it strikes the holding part. Further turning then causes the compression screw to be pulled in a lateral direction and the bone parts to thus be positioned against one another. In this case, it has proven disadvantageous, however, that a degree of compression is not limited and that the degree of compression is at the discretion of the medical professional. Particularly when older people are being treated, the problem can occur that the bone screw easily rips out of a previously damaged, and therefore no longer intact, bone part if the compression is too great. Regardless of this, a compression that is too great can also result in the bone not healing optimally.

From AT 524767 A4, an alternative device for treating a bone fracture has become known, which alternative device corresponds to the type named at the outset. In a device of this type, there is also an interaction between a bone screw and a further screw, in this case an adjusting screw. The bone screw is mounted in the opening of the holding part such that it can be moved laterally. Using the adjusting screw, a medial movement and a rotation of the bone screw are first blocked. However, a lateral movement of the bone screw is possible to a predetermined extent, which is determined by a stop of the adjusting screw that is positioned against the bone screw at an angle of a few degrees, thus contacting the bone screw obliquely. This play can be adjusted within limits of 0 mm to 12 mm, for example. During the healing process, it is thus possible that the second, medial bone part, into which the bone screw has been screwed, can respond to acting forces within predefined limits. Different constellations can thus be taken into consideration, depending on patient-specific parameters such as height, weight, bone density, and/or the type of fracture. A device according to AT 524767 A4 enables, if the mobility of the bone screw is medially blocked, a targeted setting of the lateral mobility thereof. It is not possible, however, to apply an additional tensile force to the medial bone part if it were desirable to do so.

This is addressed by the invention. The object of the invention is to further develop device of the type named at the outset such that, where necessary, a compression can also be produced.

This object is attained if, with a device of the type named at the outset, an adapter that can be fixed in the bone screw such that it can be laterally released is provided, which adapter forms a barrier for the adjusting screw in the event of lateral movement of the adjusting screw.

With the invention, the advantage is in particular obtained that a desired compression can now also be actively set to the desired extent. This can be achieved with a relatively simple design, since only one adapter is necessary which, in the event of lateral movement of the adjusting screw, normally when the same is being unscrewed, constitutes a barrier so that the adjusting screw comes to rest against the adapter. If the adjusting screw then continues to be laterally moved, typically by unscrewing the adjusting screw on the thread in the opening, the adjusting screw moves the adapter, and therefore also the bone screw connected to the adapter, in tandem in a lateral direction so that the bone screw can be laterally moved and a positioning or compression of the two bone parts can thus take place. Via the adjusting screw, which itself is positioned such that, after a lateral sliding of the bone screw, it allows said bone screw to rest against it if necessary, a closer positioning, and where necessary compression, of the broken bone parts can also take place or be produced in interaction with the adapter, so that the functionality of the device is significantly enhanced.

Within the scope of the invention, the holding part is preferably embodied to be longitudinally extended. The holding part can be a bone plate or a nail, in particular an intramedullary nail.

The holding part has at least one opening for the bone screw. The opening can be designed such that both the bone screw and the adjusting screw are guided through the opening and are thus positioned in the opening as a unit. However, it is also possible that separate openings are provided for the bone screw on the one hand and the adjusting screw on the other hand. If a single, shared opening is provided for the bone screw and the adjusting screw, said opening is typically designed such that the opening is embodied to be threadless in the contact region of the bone screw, so that the bone screw can slide in the opening. In the region of the adjusting screw, on the other hand, a thread is provided so that the adjusting screw can be screwed into the opening. If a single opening for the bone screw and the adjusting screw is provided, a cross section of the opening can be embodied to roughly pear-shaped. The cross sectional shape results from two arc-shaped opening elements which transition into one another, in order to form an opening for both the bone screw and the adjusting screw. One or both of the arcs can cover an angle of more than 180°, preferably more than 270°. The two opening elements transition into one another so that a single opening is present. The larger arc-shaped opening element is provided for the bone screw, which carries the greater load, whereas the adjusting screw is primarily, aside from the compression mechanism, provided for blocking a medial movement and for the targeted setting of a lateral movement of the bone screw and is therefore embodied with a smaller diameter, for which the second arc-shaped opening element is used.

Particularly if the holding part is longitudinally extended, as is the case for bone nails or bone plates, additional openings can be provided to accommodate additional screws with which the holding part can be fixed to the first bone part without penetrating or contacting the second bone part.

It is preferred that the adapter region of a first adapter end comprises an external thread and the bone screw comprises at a lateral bone screw end an internal thread which interacts with the external thread. In this case, the adapter can easily be releasably fastened on or in the bone screw. The adapter is thereby beneficially embodied such that it is widened relative to the bone screw in a region spaced apart from the fastening region, so that a barrier is formed for the adjusting screw. The adapter can, for example, have a cross section that is approximately 2% to 15%, for example 3% to 10%, wider than that of the bone screw.

In principle, the adapter can be embodied in any desired manner, so long as it is ensured that the adapter can form a barrier for the adjusting screw in the event of lateral movement of the same. Since the adapter must first be fastened and then released again during use, it is preferred for the sake of good handling, however, that the adapter is embodied as a longitudinally extended pin. In this case in particular, the adapter can be embodied with a tip in a region of the first adapter end, which tip is arranged in front of the external thread when viewed in a lateral direction. With the tip, which is embodied to be narrower than the external thread of the adapter, the adapter can be easily introduced into the bone screw, especially when embodied in pin form. For this purpose, the bone screw has a suitable opening. In particular, the bone screw can be embodied with an opening that runs centrally, which is normally necessary anyway.

At a lateral adjusting screw end, the adjusting screw can be embodied with a socket for a wrench or the like. As a result, the adjusting screw can be easily operated, namely both in terms of the setting of a lateral play of the bone screw and a positioning or compression of the two broken bone parts.

It is beneficial if the adjusting screw is embodied with a widened collar at the lateral adjusting screw end. The collar is multifunctional. On the one hand, the collar forms a stop surface for the bone screw, in order to limit the lateral mobility thereof during a healing process. On the other hand, during a positioning or compressing of the two bone parts, the collar serves as a stop that comes to rest against a projection of the adapter and thus initiates the explained mechanism of a targeted, active lateral movement of the bone screw.

The bone screw can be embodied with a groove in which the adjusting screw engages for the purpose of limiting, in particular preventing, a rotation of the bone screw. Furthermore, the adjusting screw can be positioned against the bone screw at an angle and engage in a groove of the bone screw that is embodied to be laterally tapered, in order to block a medial movement of the bone screw.

Aside from this, the bone screw and the adjusting screw and the openings can in particular be embodied as described in AT 524767 A4, the contents of which are hereby incorporated in their entirety.

Additional features, advantages, and effects of the invention follow from the exemplary embodiments described below. In the drawings which are thereby referenced:

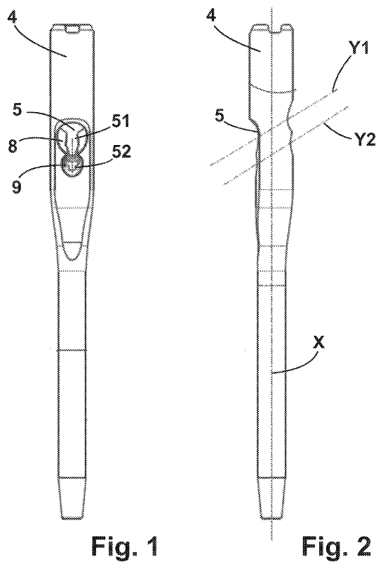

FIG. 1 shows an intramedullary nail;

FIG. 2 shows the intramedullary nail from FIG. 1 in a further view;

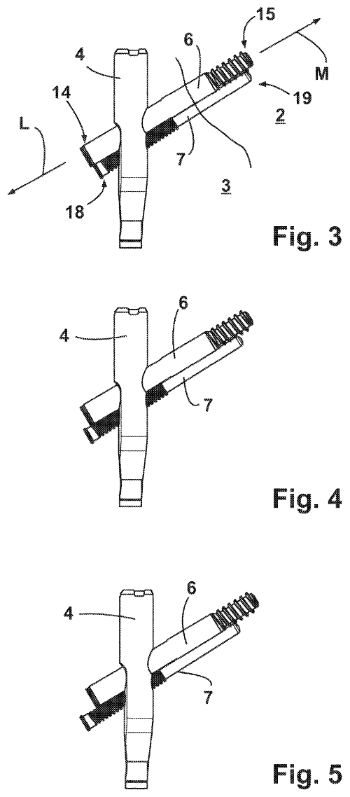

FIG. 3 shows an intramedullary nail and an adjusting screw with a bone screw and a schematic illustration of the interaction of an adjusting screw with a bone screw in a first position;

FIG. 3 shows a further schematic illustration of the interaction of an adjusting screw with a bone screw in a second position;

FIG. 5 shows a further schematic illustration of the interaction of an adjusting screw with a bone screw in a third position;

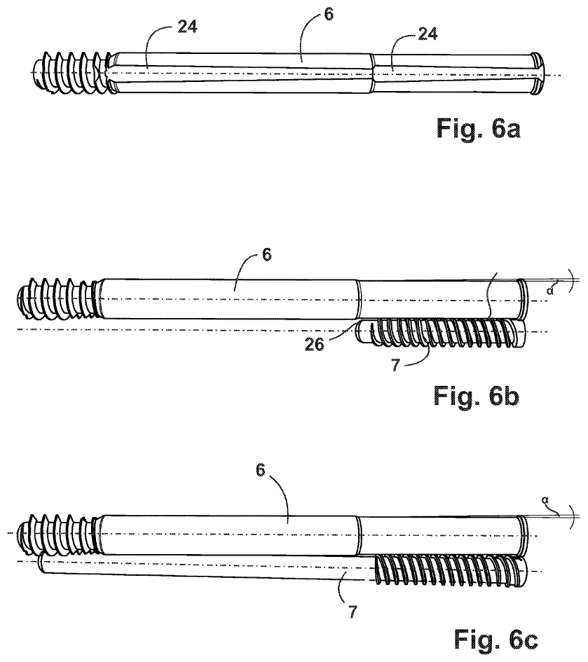

FIG. 6a through FIG. 6c show a bone screw with grooves and an interaction of the bone screw with bone screws having different lengths;

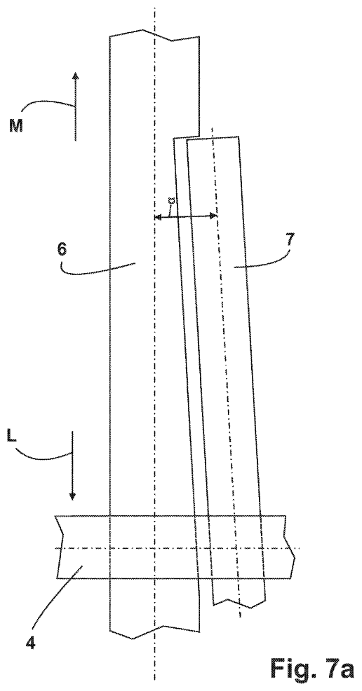

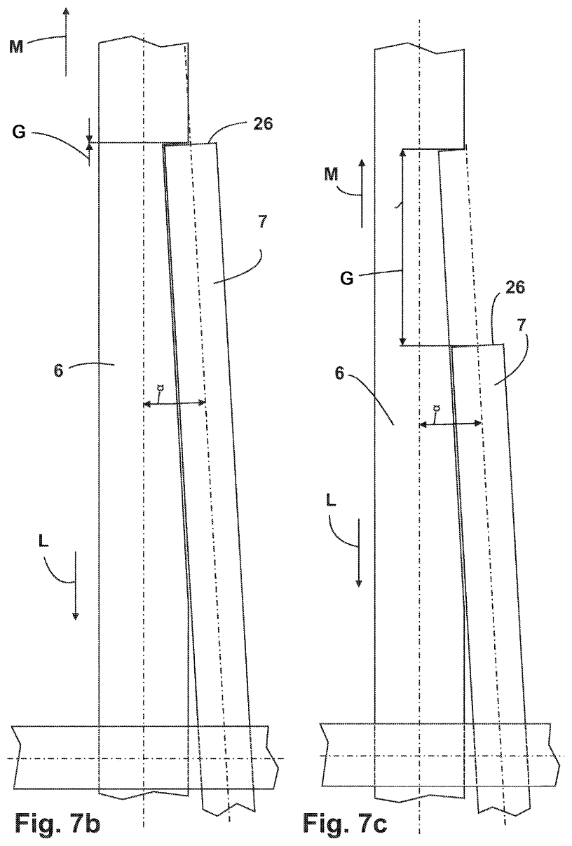

FIG. 7a through FIG. 7c show schematic illustrations of a unit comprising an intramedullary nail and a bone screw and an adjusting screw, wherein the adjusting screw defines a differing play;

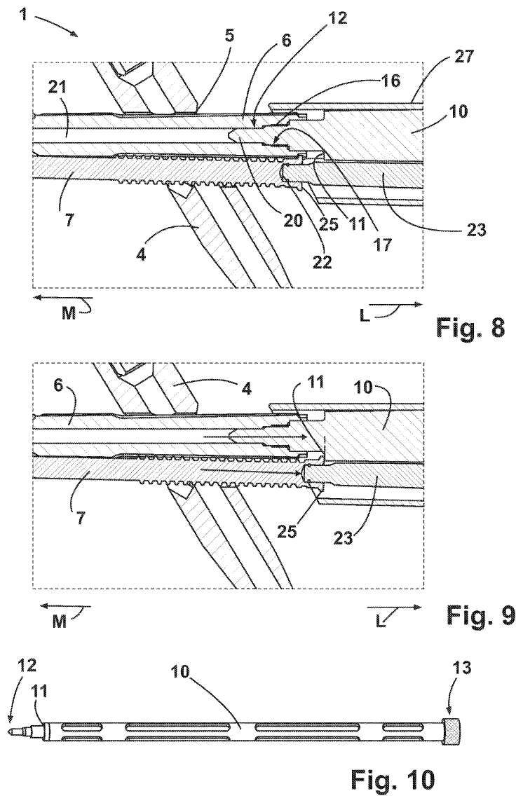

FIG. 8 shows a section of a unit comprising a bone screw and an adjusting screw and an adapter;

FIG. 9 shows the unit from FIG. 8 after a movement of the adjusting screw;

FIG. 10 shows an adapter.

In FIG. 1 and FIG. 2, a holding part 4 is illustrated. The holding part 4 is embodied as a bone nail, in particular an intramedullary nail. The holding part 4 could also be embodied as a bone plate, however, even though an embodiment of the holding part 4 as a bone nail is preferred within the scope of the invention.

The holding part 4 or intramedullary nail is, as can be seen in FIG. 1 and FIG. 2, embodied to be longitudinally extended with a longitudinal axis X. The intramedullary nail has an opening 5 with a first transverse axis Y1. The first transverse axis Y1 can, for example, form an angle of 30° to 40° with the longitudinal axis X. In the case of a bone plate as a holding part 4, an analogous opening 5 is provided. As can be seen from FIG. 1, the opening 5 is embodied with two arc-shaped segments 51, 52. An upper, first arc-shaped segment 51 is provided for a bone screw 6. An arc-shaped segment 52, positioned therebelow, of the opening 5 is provided for an adjusting screw 7. The two segments 51, 52 of the opening 5 transition into one another so that a single opening 5 for both screws is formed. The are segments thereby respectively cover more than 270°. The segment of the opening 5 for the bone screw 6 is, in cross section, larger than that for the adjusting screw 7. This results from the fact that the bone screw 6 absorbs forces and is therefore embodied to be more robust, whereas the adjusting screw 7 is used for a targeted control of the mobility of the bone screw 6 and, accordingly, can be embodied to be less robust.

The opening 5 can also be embodied such that the larger opening segment for the bone screw 6 is positioned on the bottom and the smaller opening segment for the adjusting screw 7 is positioned on the top.

The opening 5 comprises on the inner side a surface 8 that is embodied to be smooth in that region in which the bone screw 6 is or has been positioned. As a result, the bone screw 6 can slide in the opening 5 to the extent that a medial or lateral movement is not blocked. In the region of the smaller opening segment for the adjusting screw 7, a threated surface 9 is provided in which the adjusting screw 7 engages in a manner that will be explained below. The unit thus comprises, in addition to the holding part 4, which is embodied as an intramedullary nail in FIG. 1 and FIG. 2, the bone screw 6 and the adjusting screw 7. A second transverse axis Y2, which coincides with a longitudinal axis of the adjusting screw 7 or runs parallel thereto in an offset manner, forms with the first transverse axis Y1 an angle that is different than 0°.

An assembled unit is illustrated in FIG. 3 in a first position. In addition to the holding part 4, once again present as an intramedullary nail, the inserted bone screw 6 can be seen. The bone screw 6 generally comprises at a medial bone screw end 15 a thread which is used to fasten the bone screw 6 in a first bone part 2, for example a femoral head. The intramedullary nail is fastened in a different, second bone part 3 that is no longer in connection with the first bone part 2. In FIG. 3, a fracture line is schematically indicated between the two bone parts 2, 3. In the lateral direction L, the bone screw 6 is embodied to be essentially smooth after the medially arranged thread, but comprises recessed grooves 24. Said grooves 24 are widened towards a medial bone screw end 15 and, viewed in the opposite direction, tapered towards a lateral bone screw end 14. Thus, a lateral play for the bone screw 6 can be set in that, as illustrated in FIG. 3 through FIG. 5, due to the oblique positioning of the adjusting screw 7 the groove 24, a medial blocking occurs and a maximum movement for the bone screw 6 in the lateral direction L up to a stop 26 of the adjusting screw 7 is defined. The adjusting screw 7 comprises a lateral adjusting screw end 18 and a medial adjusting screw end 19. The farther the lateral adjusting screw end 18 is moved in the lateral direction L, the more lateral play the bone screw 6 has.

As noted, the adjusting screw 7 can engage in the grooves 24. The grooves 24 of the bone screw 6 and the interaction of the screws can be seen in particular in FIG. 6a through FIG. 6c. FIG. 6a shows a bone screw 6a with two grooves 24. In FIG. 6b and Fig. FIG. 6c, the corresponding arrangements are illustrated for two adjusting screws 7 having different lengths. The respective adjusting screw 7 comes to rest in one of the grooves 24. Due to the conically widened and tapered embodiment of the grooves 24, the bone screw 6 can be passively moved in the lateral direction L by occurring forces, until said bone screw 6 comes to rest against a stop surface, if necessary also multiple stop surfaces, of the adjusting screw 7. A movement in the medial direction M is, however, not possible because of the contact of the adjusting screw 7. A rotation is also inhibited at least to a large extent, advantageously entirely prevented. The interaction of the bone screw 6 and adjusting screw 7 thus results in a fixing of a medial mobility of the bone screw 6 and the rotation thereof with a simultaneous possibility of movement in the lateral direction L, provided that a corresponding play has been set using the adjusting screw 7.

For a beneficial interaction between the holding part 4, bone screw 6, and adjusting screw 7, it is favorable if the adjusting screw 7 is positioned against the bone screw 6 at a relatively small angle α, as is illustrated by way of example in FIG. 6b.

For this purpose, the corresponding longitudinal axes of the bone screw 6 and of the adjusting screw 7 are arranged such that they run towards one another in the medial direction M at an angle α, which can, for example, be in the range of 1° to 5°. The opening 5 is suitably embodied for this purpose.

In FIG. 7a through FIG. 7c, different constellations are illustrated in a highly schematized manner, in which a movement of the bone screw 6 in the lateral direction L is defined by the adjusting screw 7 and the stop 26. A gap G between the bone screw 6 and the adjusting screw 7 can be set so that, during a healing process, a movement of the bone screw 6 in the lateral direction L is possible depending on patient-specific parameters.

Normally, a play between at least one stop 26 of the adjusting screw 7 and a corresponding stop of the bone screw 6 is approximately 0 mm, 5 mm, or 10 mm. A corresponding play of 0 mm to 10 mm sufficiently covers the necessary variability with respect to patients.

The unit, explained above, of the holding part 4, bone screw 6, and adjusting screw 7 can be provided with an additional functionality if, in a device 1 which is illustrated as a section in FIG. 8 and FIG. 9, an adapter 10 is provided which forms a barrier 11 when the adjusting screw 7 is moved laterally. This is explained with the aid of FIG. 8 and FIG. 9. FIG. 8 and FIG. 9 show a holding part 4 embodied as an intramedullary nail, in the opening 5 of which a bone screw 6 and an adjusting screw 7 are arranged in the previously explained manner. The adjusting screw 7 subsequently limits a lateral movement of the bone screw 6 and prevents a medial movement of the same and a rotation of the bone screw 6. Additionally, an adapter 10 is provided which can be releasably fixed in the bone screw 6. For this purpose, the adapter 10 comprises in the region of a first adapter end 12 an external thread 16 which interacts with an internal thread of the bone screw 6 so that the adapter 10 can be guided into the bone screw 6 embodied with a central opening 21 and can be releasably fixed therein. For this purpose, the adapter 10 can also comprise at the first adapter end 12 a tip 20 which enables an insertion of the adapter 10 into the central opening 21 of the bone screw 6. Towards a second adapter end 13, as can be seen in FIG. 10, the adapter 10 is, from the viewing direction of the external thread 16, first embodied with a constant cross section that widens, so that the adapter 10 is in this region embodied with a barrier 11 present as a projection, which, as can be seen, can extend over the remaining length of the adapter 10; however, this is not mandatory. The barrier 11 is embodied to interact with the adjusting screw 7 for the purpose of positioning the second bone part 2 against the first bone part 1. To do so, the adjusting screw 7 is screwed out of the opening 5 using a wrench 23. To accommodate the wrench 23, the adjusting screw 7 has a suitable socket 22. When the adjusting screw 7 is screwed out of the opening 5, said screw initially has a free space before the adjusting screw 7 strikes the barrier 11 of the adapter 10. Since the adjusting screw 7, as noted, is positioned at an angle to the bone screw 6, the barrier 11 is embodied to sufficiently project so that a collar 25, or if necessary a different stop surface, of the adjusting screw 7 can come to rest against the barrier 11, as is illustrated in FIG. 9. Once this has occurred, the adjusting screw 7 moves the adapter 10, and thus ultimately also the bone screw 6, in tandem in the lateral direction L, so that the bone screw 6 slides to the lateral side in the opening 5, and therefore moves the second bone part 2 in the direction of the first bone part 1, as is indicated by the arrows. Once a desired positioning, if necessary compression, has been achieved, the adapter 10 can be released from the bone screw 6. The adjusting screw 7 can then, if necessary, be moved again in the medial direction M, or screwed in, in the opening 5 in order to limit a lateral play of the bone screw 6.

In FIG. 10, an adapter 10 as is expedient within the scope of the invention is illustrated. Because of the elongated embodiment of the adapter 10, it can be easily handled and, in particular, can span the distance of a tissue sleeve 27, which can be seen in FIG. 8, without difficulty.

A device according to the invention expands the range of possibilities for a unit of a holding part 4, bone screw 6, and adjusting screw 7 since, with a simultaneous limitation of a medial movement and of a rotation movement and a targeted setting of a lateral movement, a tensile force can also be applied to the bone screw 6.

1. A device for treating a bone fracture, in particular a femur fracture such as a femoral neck break, comprising:
a holding part which can be fastened in or on a first bone part, wherein the holding part has at least one opening through which a bone screw and an adjusting screw are guided,
wherein the opening comprises on the inner side a surface having a threaded surface in which the adjusting screw engages, with the bone screw being able to slide freely in the opening and the adjusting screw is configured to allow the bone screw to rest against at least one stop of the adjusting screw if necessary after a lateral sliding of the bone screw,
wherein an adapter that can be fixed in the bone screw such that it can be laterally released is provided, which adapter forms a barrier for the adjusting screw in the event of lateral movement of the adjusting screw.

2. The device according to claim 1, wherein the holding part is embodied to be longitudinally extended.

3. The device according to claim 1, wherein the holding part is embodied as an intramedullary nail.

4. The device according to claim 1, wherein the adapter comprises an external thread in the region of a first adapter end and the bone screw comprises at a lateral bone screw end an internal thread which interacts with the external thread.

5. The device according to claim 4, wherein the adapter is embodied with a tip in a region of the first adapter end, which tip is arranged in front of the external thread when viewed in a lateral direction.

6. The device according to claim 1, wherein the bone screw is embodied with an opening which runs centrally.

7. The device according to claim 1, wherein the adjusting screw is embodied with a socket for a wrench at a lateral adjusting screw end.

8. The device according to claim 1, wherein the adjusting screw is embodied with a widened collar at the lateral adjusting screw end.

9. The device according to claim 1, wherein the bone screw is embodied with a groove in which the adjusting screw engages for the purpose of limiting, in particular preventing, a rotation of the bone screw.

10. The device according to claim 1, wherein the adjusting screw is positioned against the bone screw at an angle and engages in a groove of the bone screw that is embodied to be laterally tapered, in order to block a medial movement of the bone screw.
